(19) United States
(12) Patent Application Publication
(10) Pub. No.: US 2024/0285273 A1
(43) Pub. Date: Aug. 29, 2024

(54) DEVICES AND METHODS FOR CLOSURE OF OPENINGS IN TISSUE

(71) Applicant: Boston Scientific Scimed, Inc., Maple Grove, MN (US)

(72) Inventors: Peter L. Dayton, Brookline, MA (US); James J. Scutti, Norwell, MA (US); Shawn Ryan, Littleton, MA (US); John T. Favreau, Spencer, MA (US)

(73) Assignee: Boston Scientific Scimed, Inc., Maple Grove, MN (US)

(21) Appl. No.: 18/656,048

(22) Filed: May 6, 2024

Related U.S. Application Data
(63) Continuation of application No. 17/010,063, filed on Sep. 2, 2020.
(60) Provisional application No. 62/895,103, filed on Sep. 3, 2019.

(51) Int. Cl. A61B 17/04; A61B 17/00; A61B 17/06

(52) U.S. Cl. CPC A61B 17/0469; A61B 17/06166; A61B 2017/00296; A61B 2017/00862; A61B 2017/00867; A61B 2017/0609; A61B 2017/061

(57) ABSTRACT

The present disclosure relates to the field of tissue closures including medical devices that close openings in tissue during a surgical procedure. In particular, the present disclosure relates to devices that transition from a constrained to an unconstrained configuration to engage tissue on opposing sides of a tissue opening, and hold opposing tissue portions together to maintain the opening in a closed configuration.

### CROSS-REFERENCE TO RELATED APPLICATIONS

This application is a continuation of and claims the benefit of the earlier filing date of U.S. patent application Ser. No. 17/010,063, filed Sep. 2, 2020, which claims the benefit of priority under 35 U.S.C. § 119 to U.S. Provisional Patent Application No. 62/895,103, filed Sep. 3, 2019, and which application is incorporated herein by reference in their entireties for all purposes. Any and all priority claims identified in the Application Data Sheet, or any correction thereto, are hereby incorporated by reference under 37 C.F.R. § 1.57.

### FIELD

The present disclosure pertains to medical devices. More particularly, the present disclosure pertains to devices, systems, and methods for closing tissue, such as a tissue defect or wound resulting from an endoscopic procedure, e.g., a mucosal resection or endoscopic submucosal dissection procedure.

### BACKGROUND

A tissue or opening or defect, for example a wound, such as a tissue defect within a body lumen resulting from endoscopic mucosal resection (EMR) or endoscopic submucosal dissection (ESD), may be large enough that it may be difficult to close on its own, or the time for it to close may be prolonged. A risk of infection may increase the longer the wound remains open.

It is with the above considerations in mind that the improvements of the present disclosure may be useful.

### SUMMARY

The present disclosure, in its various aspects, relates to tissue closure devices and methods for immobilizing and closing openings in target tissue.

Embodiments of the present disclosure may include a tissue closure system including a delivery device having a tubular element with a lumen therethrough, and a tissue closure device positioned within the lumen. The tissue closure device may be configured to transition between a first configuration when positioned within the lumen and a second configuration when positioned outside the lumen. The tissue closure device may include a first end portion configured to engage a first target tissue portion, a second end portion configured to engage a second target tissue portion, and a body portion connecting the first end portion to the second end portion. The first end portion may be engageable with the first target tissue portion when the first end portion is moved out of a distal end of the lumen. The second end portion may be engageable with the second target tissue portion when the second end portion is moved out of the distal end of the lumen. The first and second end portions may be biased toward each other to draw the first and second target tissue portions together when the tissue closure device is engaged therewith.

The first end portion, the second end portion, and the body portion may be a single piece of super elastic material. In some embodiments, the first and second end portions of the tissue closure device may be Nitinol and the body portion may be a suture. The tissue closure device may be C-shaped. The first configuration may be an expanded configuration in which the first and second end portions are separated by a first distance, while the second configuration may be a contracted configuration in which the first and second end portions are separated by a second distance, the first distance being greater than the second distance.

The tissue closure device may have an elongated shape when in the first configuration and may form a wave or loop shape when in the second configuration. The first and second end portions of the tissue closure device may each comprise an elongated shape when in the first configuration and may each form a plurality of wave segments when the tissue closure device is in the second configuration. The body portion may be a suture that connects the plurality of expanded loops of the first end portion to the plurality of wave segments of the second end portion. The suture may be positionable within an opening formed in the first and second targeted tissue portions, and the first and second end portions may be positionable to hold the first and second targeted tissue portions together.

When the tissue closure device is in the first configuration the tissue closure device may form a first U-shape in which the first and second end portions are biased apart. When the tissue closure device is in the second configuration the tissue closure device may form a second U-shape in which the first and second end portions are biased together. The first and second end portions may be biased apart by rotating the first and second end portions away from each other and disposing them within the lumen such that the first and second end portions are oriented in a direction away from a distal end of the lumen and the body portion is oriented toward the distal end of the lumen. Distal tips of the first and second end portions may each comprise a hook-shaped portion for engaging and retaining the first and second targeted tissue portions, respectively.

The tissue closure system may include a pusher slidably disposed within the lumen proximal to the tissue closure device, whereby distal movement of the pusher moves the tissue closure device distally. In some embodiments, the lumen may be configured to house a plurality of tissue closure devices. The plurality of said tissue closure devices can be provided as a clip or cartridge. A plurality of spacers may be disposed between adjacent tissue closure devices. In some embodiments, the delivery device is an endoscope, an FNA needle, or an FNB needle.

A tissue closure device is disclosed, including a first end portion configured to engage a first target tissue portion, a second end portion configured to engage a second target tissue portion, and a body portion connecting the first end portion to the second end portion. The tissue closure device may be configured to transition between a first configuration when constrained within a lumen of a delivery device, and a second configuration when the tissue closure device is moved out of a distal end of the lumen. The first end portion may be engageable with the first target tissue portion when the first end portion is moved out of the distal end of the lumen, while the second end portion may be engageable with the second target tissue portion when the second end portion is moved out of the distal end of the lumen. The first and second end portions may be biased toward each other to draw the first and second target tissue portions together when the tissue closure device is engaged therewith.

In some embodiments, the tissue closure device may be formed from a super elastic material. In some embodiments, the first and second end portions of the tissue closure device may be formed from Nitinol and the body portion may be a suture.

The tissue closure device can be C-shaped. The first configuration may be an expanded configuration in which the first and second end portions are separated by a first distance, and the second configuration can be a contracted configuration in which the first and second end portions are separated by a second distance. The first distance may be greater than the second distance. The tissue closure device may have an elongated shape when in the first configuration and may form a wave or loop shape when in the second configuration.

The first and second end portions of the tissue closure device may each comprise an elongated shape when in the first configuration. The first and second end portions may each assume a plurality of wave segments when tissue closure device is in the second configuration.

The body portion may be a suture that connects the plurality of wave segments of the first end portion to the plurality of wave segments of the second end portion. The suture may be positionable within an opening formed in the first and second targeted tissue portions, while the first and second end portions may be positionable to hold the first and second targeted tissue portions together.

When the tissue closure device is in the first configuration the tissue closure device may have a first U-shape in which the first and second end portions are biased apart. When the tissue closure device is in the second configuration the tissue closure device may have a second U-shape in which the first and second end portions are biased together. Distal tips of the first and second end portions may each comprise a hook-shaped portion for engaging and retaining the first and second targeted tissue portions, respectively.

A method for closing an opening in tissue is disclosed. The method may include positioning a lumen of a delivery device adjacent to a target tissue opening, moving a first end portion of a tissue closure device out of a distal end of the lumen to engage a first end portion of the tissue closure device with a first target tissue portion, and moving a second end portion of a tissue closure device out of the distal end of the lumen to engage the second end portion of the tissue closure device with a second target tissue portion. The method may include drawing the first and second target tissue portions together using a bias in the tissue closure device that urges the first and second end portions of the tissue closure device toward each other. For embodiments in which the first and second end portions of the tissue closure device are coupled to each other by a suture, the method further include drawing the first and second target tissue portions together using a bias in the first end portion that urges the first target tissue portion toward the second target tissue portion and using a bias in the second end portion that urges the second target tissue portion toward the first target tissue portion.

A medical device is disclosed, including a handle portion, a flexible tube portion coupled at a proximal end to the handle portion, and having an articulable distal tip; and a tissue closure device integrated into a distal tip of the flexible tube portion. The tissue closure device can be configured to transition between a first configuration when positioned within the distal tip and a second configuration when positioned outside the distal tip. The tissue closure device may include a first end portion configured to engage a first target tissue portion, a second end portion configured to engage a second target tissue portion, and a body portion connecting the first end portion to the second end portion. The first end portion can be engageable with the first target tissue portion when the first end portion is moved out of a distal tip. The second end portion can be engageable with the second target tissue portion when the second end portion is moved out of the distal tip. The first and second end portions may be biased toward each other to draw the first and second target tissue portions together when the tissue closure device is engaged therewith. In some embodiments the medical device is an endoscope including at least one of a camera, a light, and a working channel.

In some embodiments, the first end portion, the second end portion, and the body portion comprise a single piece of super elastic material. The first and second end portions of the tissue closure device may comprise Nitinol and the body portion comprises a suture. The tissue closure device can be C-shaped, and wherein the first configuration is an expanded configuration in which the first and second end portions are separated by a first distance, and wherein the second configuration is a contracted configuration in which the first and second end portions are separated by a second distance, the first distance being greater than the second distance.

The tissue closure device may have an elongated shape when in the first configuration, and wherein the tissue closure device comprises a wave or loop shape when in the second configuration. The first and second end portions of the tissue closure device can each comprise an elongated shape when in the first configuration, and wherein the first and second end portions each assumes a plurality of wave segments when the tissue closure device is in the second configuration. The body portion may comprise a suture that connects the plurality of wave segments of the first end portion to the plurality of wave segments of the second end portion, wherein the suture is positionable within an opening formed in the first and second targeted tissue portions, and wherein the first and second end portions are positionable to hold the first and second targeted tissue portions together. When the tissue closure device is in the first configuration the tissue closure device comprises a first U-shape in which the first and second end portions are biased apart, and wherein when the tissue closure device is in the second configuration the tissue closure device comprises a second U-shape in which the first and second end portions are biased together.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIG. 1A is an illustration of an example endoscope according to an embodiment of the present disclosure.

FIG. 1B is an illustration of a system being deployed from an endoscope according to an embodiment of the present disclosure.

FIG. 2A is a perspective view of a tissue closure device according to an embodiment of the present disclosure.

FIG. 2B is a perspective view of a plurality of the tissue closure devices of FIG. 2A engaged with a target tissue site according to an embodiment of the present disclosure.

FIG. 2C is a side view of the tissue closure device of FIG. 2A engaged with a target tissue site according to an embodiment of the present disclosure.

FIG. 2D is a cross-section view of the tissue closure device of FIG. 2A disposed within an example lumen of a delivery device.

FIG. 3A is a side view of a tissue closure device according to an embodiment of the present disclosure.

FIG. 3B is a cross-section view of the tissue closure device of FIG. 3A disposed within an example lumen of a delivery device.

FIGS. 3C-3G are a sequence of perspective views of the tissue closure device of FIG. 3A engaging a target tissue site.

FIG. 3H is a side view of the tissue closure device of FIG. 3A engaged with a target tissue site according to an embodiment of the present disclosure.

FIGS. 4A-4C are a series of cross-section views of a tissue closure device according to an embodiment of the present disclosure engaging a target tissue site.

FIGS. 4D-4E are cross-section views of an alternative embodiment of the tissue closure device of FIGS. 4A-4C.

FIGS. 4F-4G are cross-section views of a further alternative embodiment of the tissue closure device of FIGS. 4A-4C.

FIGS. 5A-5C are a series of cross-section views of a tissue closure device according to an embodiment of the present disclosure engaging a target tissue site.

FIG. 5D is a side view of the tissue closure device of FIG. 5A engaged with a target tissue site according to an embodiment of the present disclosure.

FIG. 6A is a perspective view of a clip of tissue closure devices according to an embodiment of the present disclosure.

FIG. 6B is a perspective view of the clip of tissue closure devices of FIG. 6A in proximity to an example endoscope.

FIG. 6C is a side view of one of the tissue closure devices of FIG. 6A engaged with a target tissue site according to an embodiment of the present disclosure.

It is noted that the drawings are intended to depict only typical or examples of embodiments of the disclosure. Accordingly, the drawings should not be considered as limiting the scope of the disclosure.

### DETAILED DESCRIPTION

Various embodiments according to the present disclosure are described below. As used herein, “proximal end” refers to the end of a device that lies closest to the medical professional along the device when introducing the device into a patient, and “distal end” refers to the end of a device or object that lies furthest from the medical professional along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The detailed description should be read with reference to the drawings, which are not necessarily to scale, depict illustrative embodiments and are not intended to limit the scope of the invention.

The disclosure is not limited to the particular embodiments described, as such may vary. The terminology used herein is for the purpose of describing particular embodiments only and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to medical devices and systems and procedures for dissecting tissues of the digestive system, it should be appreciated that such medical devices and methods may be used to dissect tissues of the abdominal cavity, gastrointestinal system, thoracic cavity, urinary and reproductive tract and the like. Moreover, a variety of medical procedures may benefit from the presently disclosed medical devices and procedures, including, for example, Endoscopic Submucosal Dissection (ESD), Peroral Endoscopic Myotomy (POEM), cholecystectomy and Video-Assisted Thorascopic Surgery (VATS) procedures. The structures and configurations, and methods of deploying, in order to stabilize, manipulate and provide a clear field of view may find utility beyond dissection.

The trend in medicine is moving from laparoscopic and open surgical procedures to miniaturized, endoscopic procedures. The Endoscopist can perform ever more complex non or minimally invasive procedures under direct visualization. Current endoscopes provide working channels to enable the use of dedicated instruments for such treatments, but they may not include the intrinsic capability to treat and manipulate tissue being accessed and examined. There exists a need for endoscopes, colonoscopes, duodenoscopes, bronchoscopes, gastroscopes, uteroscopes, or similar medical devices which possess built-in treatment capabilities. Such devices can facilitate both a broad range of procedural interventions that are becoming more prevalent in hospitals and can lead to the development of significantly more capable and complex scope designs.

Further, infection prevention controls in the clinical setting create a demand for single-use scopes which mitigate the risk of patient infection and associated serious adverse events. For example, currently commercial duodenoscopes often include distal tips having complex mechanical features. Such complexity can make it difficult to properly disinfect reusable scopes between procedures.

The disclosure pertains to medical devices, e.g., endoscopes, gastroscopes, bronchoscopes, colonoscopes, ureteroscopes, and the like, having integrated features for acquiring, manipulating, and closing openings in target tissue. Although single-use endoscopes are described herein, it is understood that embodiments of the present disclosure may be included in reusable medical devices such as endoscopes as well. The disclosed systems, methods and devices include an endoscope in which complex functionality is integrated into the design of the endoscope. Such complex functionality includes one or more of suturing, stapling, clip deployment, and the like. The disclosed systems, methods and devices can be of a single-use design that can aid in infection prevention. That is, the disclosed endoscopes, which provide solutions to complex noninvasive clinical needs, can minimize or eliminate infection risks because they can be sterile, single-use devices.

In some embodiments, the disclosed systems, methods, and devices provide practitioners the ability to close defects or to suture tissue in apposition. For example, clips can be used to close defects or suture tissue. Such clips can be single use, one clip per device arrangements. Alternatively, devices can be provided with multiple clips to enable the devices to be used for closing any of a variety of opening sizes. The disclosed systems, methods, and devices can improve closure capability using different mechanisms (i.e., clip/closure designs) while also maintaining ease of use. In some non-limiting example embodiments, tissue-to-tissue attachment can be achieved using a closure device consisting of one or more clips, or a combination of one or more clips and one or more sutures.

Embodiments include closure devices made from super-elastic material such as Nitinol, though other materials may be used, such as polymers having appropriate elastic characteristics. In some embodiments the closure devices may be delivered via an endoscope, while in other embodiments the closure devices may be delivered via a needle (e.g., fine needle aspiration (FNA) or fine needle biopsy (FNB)). The closure devices may be disposed within a lumen of the delivery device and may be delivered to the targeted tissue site in a straightened/constrained configuration. In some embodiments, the closure devices may be delivered using an endoscope having closure capabilities integrated into a distal end of the endoscope. The delivery device may deploy the closure devices beyond a distal end of the device/lumen. Upon deployment, the closure devices may assume a configuration facilitating engagement of opposing segments of target tissue.

In some embodiments, the closure devices may force or draw the opposing segments of target tissue together and may hold the segments together to promote healing. In other embodiments, the closure devices may hold opposing segments of target tissue together after the segments have been forced or drawn together using other devices.

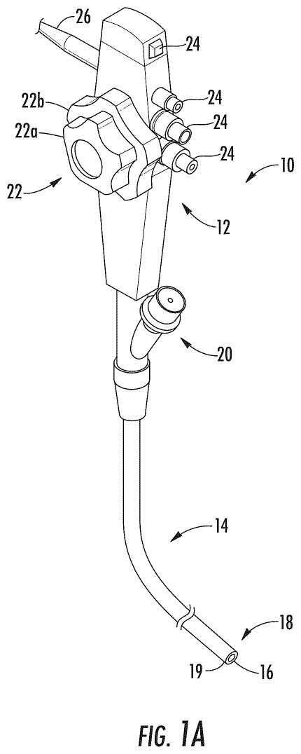

An example endoscope 10 is illustrated in FIG. 1A. The endoscope 10 may be any of a number of types of endoscopes or related medical devices usually identified by the particular anatomy desired to be reached. For example, the endoscope 10 may be any of a number of types of colonoscopes, duodenoscopes, bronchoscopes, gastroscopes, uteroscopes, or similar medical devices. The endoscope 10 may include a handpiece 12 and an elongate shaft 14 extending distally from the handpiece 12 to a distal tip 18. The shaft 14 may include a lumen defining a working channel 16 extending through the shaft 14 from a distal end 19 near the distal tip 18 of shaft 14 to an access port 20 that may be positioned in the handpiece 12 or another portion of the endoscope 10. Although the endoscope 10 is depicted with a single working channel in FIG. 1A, it can be appreciated that in other embodiments, the endoscope 10 may include multiple working channels, as desired.

In some cases, the handpiece 12 may include one or a plurality of controls 22, such as rotating knobs, which may be used to control movement of the distal tip 18 of the shaft 14 during operation. For example, a first rotating knob 22a may control up and down movement or deflection of the distal tip 18 of the shaft 14, while a second rotating knob 22b may control side-to-side movement or deflection of the distal tip 18 of the shaft 14. The handpiece 12 may also include one or a plurality of buttons 24, which may be used to activate suction or deliver fluid such as air, saline and/or water, etc. through a lumen of the endoscope 10 or perform other functions as desired. Additionally, in some cases, the handpiece 12 may include an optical cable 26 connected to an external light source (not shown). An imaging element, such as a charge coupled device (CCD) camera can be disposed at the distal tip to enable a user to visualize a working area.

As illustrated in FIG. 1B, a non-limiting example embodiment of a distal tip of an endoscope 110 is shown for use in delivering and positioning a tissue closure device 102 of the present disclosure at a target tissue 114 site. The tissue closure device 102 may be positioned within a lumen 112 of the endoscope 110. The tissue closure device 102 may be pre-loaded within the lumen 112, or it may be loaded into the lumen 112 while in the endoscope 110 is in use by using a push member to translate the tissue closure device 102 distally. In some embodiments, the endoscope 110 includes a tissue closure device 102 integrated into the distal end of the endoscope 110. The tissue closure device may be controllable via a handle at a proximal end of the endoscope 110.

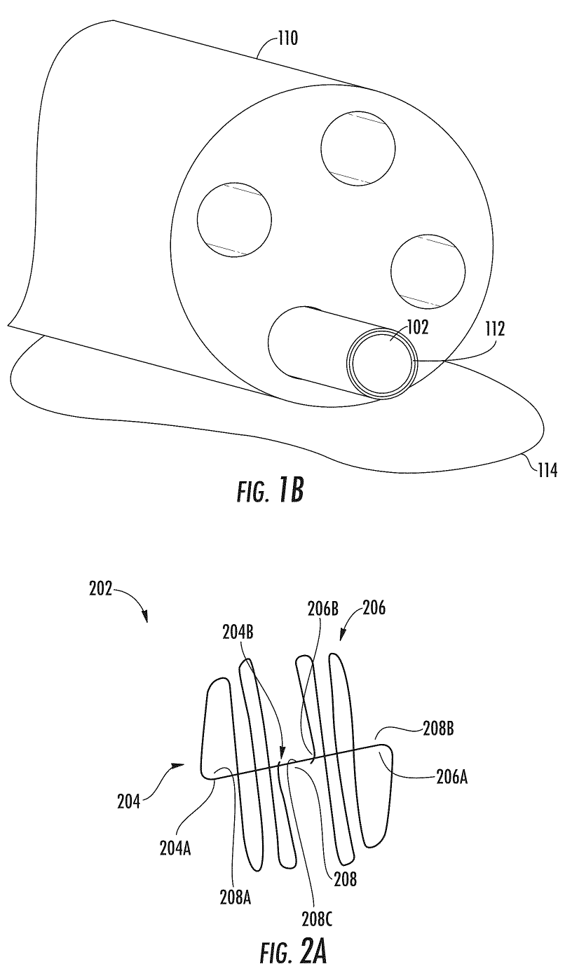

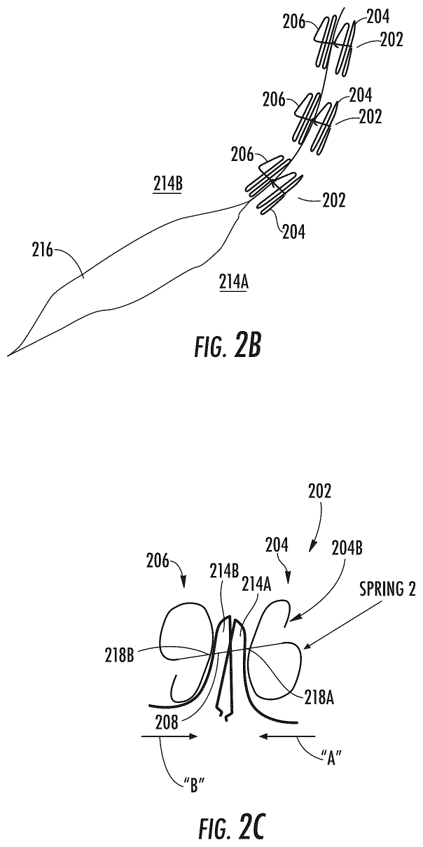

An embodiment of a tissue closure device 202 according to the present disclosure is illustrated in FIGS. 2A-2D. The tissue closure device 202 includes first and second end portions 204, 206 and a body portion 208 that connects the first end portion to the second end portion. In some nonlimiting example embodiments the first and second end portions 204, 206 comprise super-elastic wire elements such as Nitinol and the body portion 208 comprises a suture. In some embodiments, the entire tissue closure device 202 may be formed of Nitinol or other shape memory material. The first end portion 204 can have a first end 204A coupled to a first end 208A of the body portion 208, and a second end 204B that is a free end. The second end portion 206 can have a first end 206A coupled to a second end 208B of the body portion 208, and a second end 206B that is a free end. The first and second end portions 204, 206 may be coupled to the body portion 208 using any of a variety of appropriate connection technologies, including adhesives, crimping, swaging, and the like. In some embodiments, the first and second end portions 204, 206 and the body 208 may be a single component.

The first and second end portions 204, 206 can each comprise flexible wire elements that form a plurality of segments that form a plurality of wave segments when in the unconstrained condition illustrated in FIGS. 2A-2C. As will described in greater detail later, the first and second end portions 204, 206 may be constrained to assume an extended, generally straightened, configuration (see FIG. 2D) when disposed within the endoscope 110. Upon delivery, the first and second end portions 204, 206 may naturally expand to the configuration shown in FIGS. 2A-2C to engage first and second target tissue portions 214A, 214B and to press those tissue portions together.

The plurality of wave segments of the second ends 204B, 206B of the first and second end portions 204, 206 may be positioned such that the second ends are disposed adjacent to a central portion 208C of the body portion 208 when the tissue closure device 202 is in the unconstrained condition. The first and second end portions 204, 206 may be comprised of a resilient material such that the first and second end portions function as spring elements. By positioning the second ends 204B, 206B of the first and second end portions 204, 206 adjacent to the central portion 208C of the body portion, the plurality of wave segments of each of the first and second end portions 204, 206 comprise spring elements that are biased toward the center of the tissue closure device 202 (i.e., as shown in FIG. 2C, the first end portion 204 biases a first target tissue portion 214A in the direction of arrow “A” while the second end portion 206 biases a second target tissue portion 214B in the direction of arrow “B”). Thus, when the first end portion 204 is engaged with a first target tissue portion 214A and the second end portion 206 is engaged with a second target tissue portion 214B, the spring bias in the plurality of wave segments urge the first and second target tissue portions together to seal an opening 216 in the tissue.

In the illustrated embodiment the body portion 208 is disposed through respective openings 218A, 218B in the first and second target tissue portions 214A, 214B. In some embodiments the respective openings 218A, 218B may be formed by the second end 204B of the first end portion 204, which may penetrate the first and second target tissue portions 214A, 214B as it is deployed from the lumen 112 (FIG. 1) of the endoscope 110 or needle. Thus, the second end 204B of the first end portion 204 may have a needle-like tip suitable for penetrating tissue, although the size and rigidity of the first end portion itself may be sufficient to penetrate the first and second target tissue portions 214A, 214B.

The tissue closure device 202 may be delivered to the target tissue site in a first configuration, which in one embodiment is a generally elongated configuration (FIG. 2D). As can be seen, in this configuration the first and second end portions 204, 206 are in a relatively compressed, or constrained, elongated configuration when disposed within the lumen 112 of the endoscope 110 or needle. In addition, the tissue closure device 202 can be positioned so that the second end 204B of the first end portion 204 is positioned adjacent a distal end 112A of the lumen 112. The second end 204B may also be oriented along a longitudinal axis X-X of the lumen 112 to enable the user to direct the second end 204B toward the target tissue site.

For embodiments in which the tissue closure device 202 is used with an endoscope 110 (FIG. 1), the second end 204B of the first end portion 204 may be deployed from a distal end 112A of the lumen 112 so that the second end 204B engages and penetrates the first and second target tissue portions 214A, 214B. Deployment can be achieved using an appropriate pusher (not shown) disposed behind the tissue closure device 202 within the lumen 112. The user can apply an appropriate force to the pusher to move the second end 204B of the first end portion 204 out of the distal end 112A of the lumen 112 and into engagement with the first and second target tissue portions 214A, 214B until the first end portion 204 passes through both tissue portions. In some embodiments, the lumen 112 may be retractable in a proximal direction, to expose the closure device 202. In some embodiments, the closure device 202 may be housed within a sheath that holds the closure device in a substantially straight, constrained configuration. The sheath may include a sharp distal tip for penetrating the first and second target tissue portions 214A, 214B. Once the sheath penetrates the first and second target tissue portions 214A, 214B, the sheath may be retracted in a proximal direction relative to the closure device 202, exposing the closure device 202, and releasing the closure device 202 to a deployed, unconstrained configuration (e.g., see FIG. 2C). FIG. 2C shows the first end portion 204 disposed adjacent the first target tissue portion 214A and the second end portion 206 disposed adjacent the second target tissue portion 214B, with the body portion 208 spanning the two. As can be seen, the plurality of wave segments of the second ends 204B, 206B of the first and second end portions 204, 206 engage the first and second target tissue portions 214A, 214B and press the tissue portions together.

For embodiments in which the tissue closure device 202 is deployed using a needle (not shown), the needle may be used to penetrate the first and second target tissue portions 214A, 214B. In such arrangements, once the needle has been advanced through the first and second target tissue portions 214A, 214B, the second end 204B of the first end portion 204 may be deployed sequentially from a distal end 112A of the lumen 112 of the needle until the first end portion 204 is positioned beyond the first target tissue portion 214A. The needle may then be retracted until the second end portion 206 is positioned beyond the second target tissue portion 214B. Again, the plurality of wave segments of the second ends 204B, 206B of the first and second end portions 204, 206 can engage the first and second target tissue portions 214A, 214B and press the tissue portions together.

As shown in FIG. 2B, a plurality of tissue closure devices 202 may be used to seal an opening 216 in the tissue. Where a plurality of tissue closure devices 202 are employed, such tissue closure devices may be pre-loaded within the lumen 112 in the endoscope 110 or needle and sequentially deployed using a pusher disposed behind the pre-loaded tissue closure devices. Alternatively, individual tissue closure devices 202 may be loaded into the lumen 112 and deployed individually.

In the illustrated embodiment the tissue closure comprises first and second end portions 204, 206 made from a super-elastic material such as Nitinol coupled to a body portion 208 comprising a suture. It is contemplated that the first and second end portions 204, 206 could alternatively comprise elastic tubular elements, with the body portion 208 comprising a suture disposed through an internal diameter of the tubular elements. The suture may, in some embodiments, be a quill-type suture and/or may have surface features that enable the suture to lock to an inner surface of the tubular elements. When constrained and nearly straight (i.e., the first configuration, which may be a delivery configuration), the body portion 208 may be free to move within the first and second end portions 204, 206. When the tissue closure device 202 is deployed from the lumen 112 of the endoscope 110 or needle, an angulation of the first and second end portions 204, 206 may capture the body portion 208 via the quills or other surface features. Such an arrangement would allow for variable lengths of the body portion 208, since the second end portion 206 would allow the body portion 208 to move within the second end portion until the second end portion is deployed. Friction and/or interference between the second end portion 206 and the body portion 208 may lock the body portion in place. For example, the surface features could be disposed on the suture 208, on an inner surface of the elastic tubular elements 204, 206, or both. Small, pushed in, sections of the tubular wall may greatly increase the friction when the tube bends into its final configuration or catch the quills on that type of suture. Alternatively, an outside wall of the tubes could be cut (no indentation) to make small windows in the tube wall that would mate with a quilled suture.

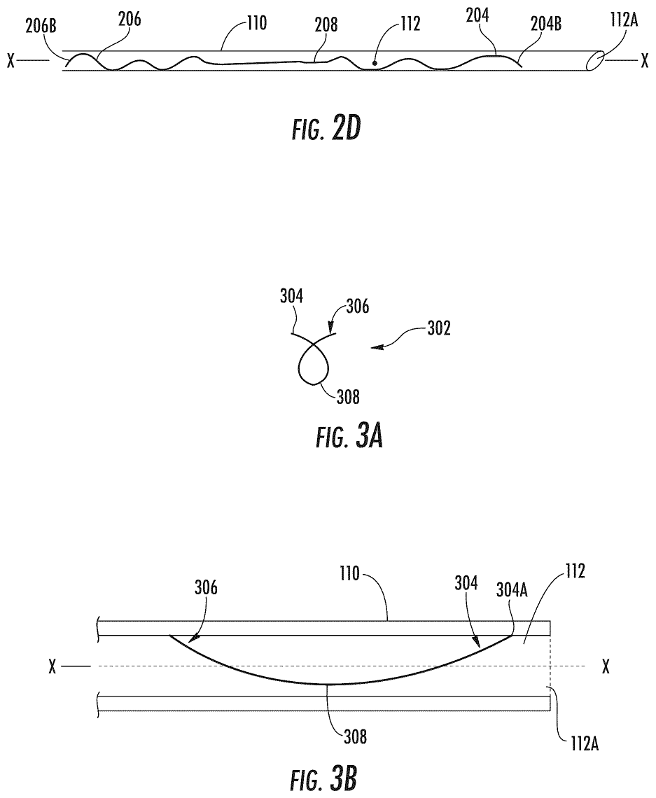

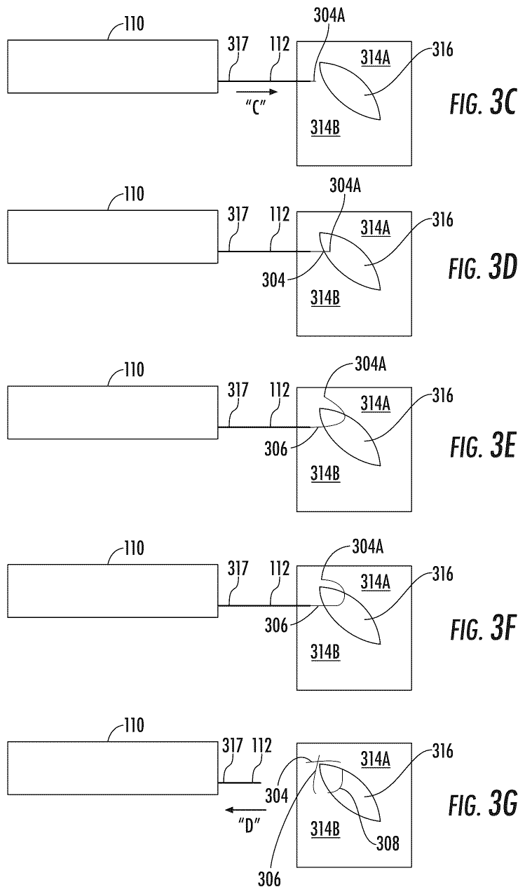

An embodiment of a tissue closure device of the present disclosure is illustrated in FIGS. 3A-3F. The tissue closure device 302 comprises first and second end portions 304, 306 and a body portion 308 that connects the first end portion to the second end portion. In some nonlimiting example embodiments the first and second end portions 304, 306 and the body portion 308 comprise a single super-elastic wire element such as Nitinol or other shape memory material.

The tissue closure device 302 can be formed so that the first and second end portions 304, 306 form a loop when the tissue closure device 302 is in an unconstrained condition illustrated in FIG. 3A. As mentioned, the first and second end portions 304, 306 and the body portion 308 may be comprised of a super-elastic material such that the first and second end portions (and the body portion) function as a spring, causing the first and second end portions to be biased toward each other. As will be described, when the first end portion 304 is engaged with a first target tissue portion 314A and the second end portion 306 is engaged with a second target tissue portion 314B, the spring bias of the first and second end portions 304, 306 urges the first and second target tissue portions together to seal an opening 316 in the tissue.

The tissue closure device 302 may be delivered to a target tissue site in a first configuration, which in one embodiment is a generally elongated configuration as shown in FIG. 3B. As can be seen, the first and second end portions 304, 306 are in a relatively compressed, elongated configuration when disposed within the lumen 112 of the distal end of the endoscope 110 or needle. In addition, the tissue closure device 302 can be positioned so that distal tip 304A of the first end portion 304 is positioned adjacent a distal end 112A of the lumen 112. The tissue closure device 302 may also be generally oriented along the longitudinal axis X-X of the lumen 112 to enable the user to direct the distal tip 304A of the tissue closure device 302 toward the target tissue site.

Referring now to FIGS. 3C-3F, deployment of the tissue closure device 302 will be described in greater detail. In FIG. 3C, the endoscope 110 can be positioned adjacent an opening 316 between the first and second target tissue portions 314A, 314B. A sheath 317 housing the lumen 112 may be advanced in the direction of arrow “C” toward the first and second target tissue portions 314A, 314B so that the distal tip 304A is directed at the second target tissue portion 314B. A pusher (not shown) disposed within the lumen 112 may be used to move the tissue closure device 302 so that the distal tip 304A engages the second target tissue portion 314B. The distal tip 304A may penetrate second target tissue portion 314B. As the tissue closure device 302 is moved further out of the lumen 112, the distal tip 304A extends across the opening 316 (FIG. 3D) until the distal tip engages and penetrates the first target tissue portion 314A (FIG. E). As can be seen, the spring bias in the tissue closure device 302 may cause the distal tip 304A (and the first end portion 304) to loop back toward the second end portion 306. Further advancement of the tissue closure device 302 results in the first end portion 304 crossing back over the second end portion 306 (FIGS. 3F and 3G). The result, illustrated in FIG. 3H, is the first and second end portions 304, 306 crossing each other due to the spring bias in the tissue closure device 302, which presses the first and second target tissue portions 314A, 314B together.

In some embodiments, the distal tip 304A of the first end portion 304 may have a needle-like tip suitable for penetrating tissue, although the size and rigidity of the first end portion itself may be sufficient to penetrate the first and second target tissue portions 314A, 314B.

For embodiments in which the tissue closure device 302 is used with a distal end of an endoscope 110, the distal tip 304A of the first end portion 304 may be deployed from a distal end 112A of the lumen 112 of the endoscope so that the distal tip engages and penetrates the first and second target tissue portions 314A, 314B. Deployment can be achieved using an appropriate pusher (not shown) disposed behind the tissue closure device 302 within the lumen 112. The user can apply an appropriate force to the pusher to move the distal tip 304A of the first end portion 304 out of the distal end 112A of the lumen 112 and into engagement with the first and second target tissue portions 314A, 314B until the first end portion 304 passes through both tissue portions.

For embodiments in which the tissue closure device 302 is deployed using a needle (not shown), the needle may be used to penetrate the first and second target tissue portions 314A, 314B. In such arrangements, once the needle has been advanced through the first and second target tissue portions 314A, 314B, the first end portion 304 may be deployed sequentially from a distal end of the needle until the first end portion 304 is positioned beyond the first target tissue portion 314A. The needle may then be retracted until the second end portion 306 is positioned beyond the second target tissue portion 314B. The spring bias in the first and second end portions 304, 306 can engage the first and second target tissue portions 314A, 314B and press the tissue portions together.

Although not shown, it will be appreciated that a plurality of tissue closure devices 302 may be used to close an opening 316 in the tissue. Where a plurality of tissue closure devices 302 are employed, such tissue closure devices may be pre-loaded within the lumen 112 in the endoscope 110 or needle and may be sequentially deployed using a pusher disposed behind the pre-loaded tissue closure devices. Alternatively, individual tissue closure devices 302 may be loaded into the lumen 112 and deployed individually as required.

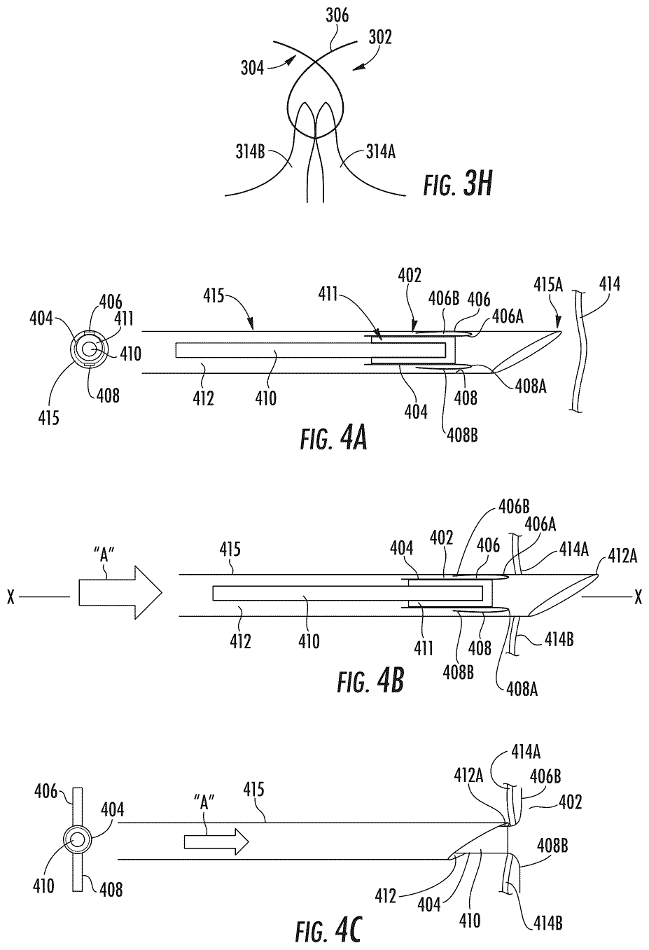

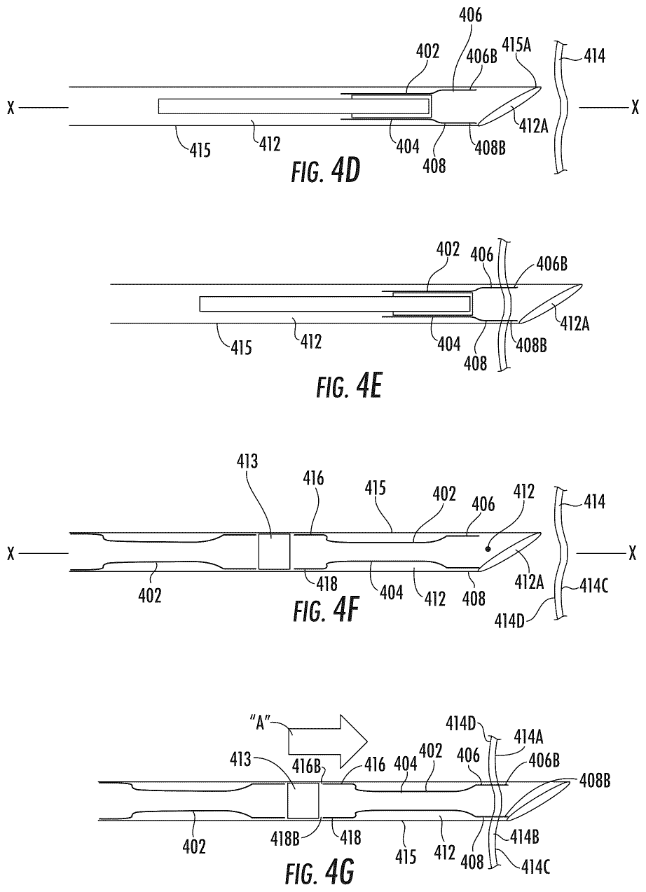

An embodiment of a tissue closure device of the present disclosure, is illustrated in FIGS. 4A-4C. The tissue closure device 402 comprises a body portion 404 which may be a tubular element, and first and second end portions 406, 408 coupled thereto. In some embodiments, the body portion 404 and first and second end portions 406, 408 can be different portions of the same piece. For example, the body portion 404 may be a piece of tube (e.g., hypotube) with two flaps comprising the first and second end portions 406, 408. In addition, more than two end portions could also be used. The body portion 404 may also be coupled to a suture portion 410 along an inner or outer surface thereof. The body portion 404 may be coupled to the suture portion using any appropriate connection mechanism, such as gluing, crimping, swaging, and the like. In the illustrated embodiment the body portion 404 is coupled to the suture portion 410 via an adhesive 411. In some nonlimiting example embodiments the first and second end portions 406, 408 are tab elements made from a super-elastic material such as Nitinol. The tabs may be provided in any of a variety of appropriate shapes and sizes.

The first and second end portions 406, 408 may each have a proximal portion 406A, 408A and a distal portion 406B, 408B. The proximal portions 406A, 408 may be coupled to the body portion 404, while the distal portions 406B, 408B may extend away from the proximal portions when the tissue closure device 402 is in the unconstrained condition (FIG. 4C).

As mentioned, the first and second end portions 406, 408 may be made from a super-elastic material so that the first and second end portions function as a spring. In some embodiments the proximal portions 406A, 408A of the first and second end portions 406, 408 are biased away from their respective distal portions 406B, 408B so that when the first and second end portions 406, 408 are engaged with first and second target tissue portions 414A, 414B, the distal portions 406B, 408B extend away from the proximal portions 406A, 408A to hold the first and second target tissue portions to the body portion 404.

The tissue closure device 402 may be delivered to the target tissue site in a first configuration, which is illustrated in FIGS. 4A and 4B. In this configuration the tissue closure device 402 is disposed within the lumen 412 of a needle 415. As can be seen, the distal portions 406B, 408B are folded backward so that they lie approximately parallel to an outer surface of the body portion 404. The tissue closure device 402 can be positioned so the proximal portions 406A, 406B are positioned adjacent a distal end 412A of the lumen 412. The tissue closure device 402 may also be generally oriented along the longitudinal axis X-X of the lumen 412 to enable the user to direct the tissue closure device 402 toward the target tissue site.

To engage the tissue closure device 402 with target tissue, the needle 415 is positioned adjacent a target tissue portion 414. A distal end 415A of the needle 415 may be moved into engagement with the target tissue portion 414 so that the distal end pierces the target tissue portion. A pusher (not shown) disposed within the needle 415 may be used to move the tissue closure device 402 in the direction of arrow “A” out of the lumen 412 so that the distal portions 406B, 408B of the first and second end portions 406, 408 pass through the target tissue portion. As the distal portions 406B, 408B of the first and second end portions 406, 408 pass through the target tissue portion the bias in the first and second end portions 406, 408 causes the distal portions 406B, 408B to rotate (or otherwise move) away from their respective proximal portions 406A, 408A. The distal portions 406B, 408B may thus assume the position shown in FIG. 4C in which the distal portions engage the first and second target tissue portions 414A, 414B, respectively. The needle 415 may then be removed, leaving the tissue closure device 402 coupled to the first and second target tissue portions 414A and 414B. In some embodiments the suture portion 410 may be manipulated to close an opening between the first and second target tissue portions 414A, 414B.

FIGS. 4D-4E illustrate an embodiment of the tissue closure device 402 in which the first and second end portions 406, 408 are folded forward so that they lie approximately parallel to an outer surface of the body portion 404. The tissue closure device 402 can be positioned so the distal portions 406B, 408B are positioned adjacent a distal end 412A of the lumen 412. The tissue closure device 402 may also be generally oriented along the longitudinal axis X-X of the lumen 412 to enable the user to direct the tissue closure device 402 toward the target tissue site. To engage the tissue closure device 402 with target tissue, the distal end 415A of the needle 415 may be moved into engagement with a target tissue portion 414 to pierce target tissue portion. A pusher (not shown) disposed within the needle 415 may be used to move the tissue closure device 402 in the direction of arrow “A” out of the lumen 412 so that the distal portions 406B, 408B of the first and second end portions 406, 408 pass through the target tissue portion. As the distal portions 406B, 408B of the first and second end portions 406, 408 pass through the target tissue portion the bias in the first and second end portions 406, 408 causes the distal portions 406B, 408B to rotate (or otherwise move) away from their respective proximal portions 406A, 408A. The distal portions 406B, 408B may thus couple the tissue closure device 402 to first and second target tissue portions 414A, 414B in a manner similar to that shown in FIG. 4C.

FIGS. 4F-4G illustrate an embodiment of the tissue closure device 402 in which a plurality of tissue closure devices 402 may be disposed in series within the lumen 412 of the needle 415. The tissue closure devices 402 of this embodiment may or may not be used in combination with a suture. The tissue closure devices 402 are shown separated by a spacer 413, which may allow full clip deployment without exposing (and potentially partially deploying) the subsequent clip. The spacer 413 may be made from any biocompatible material including biodegradables. The spacer 413 may be removed after the closure is complete or could be allowed to pass naturally through the gastro-intestinal tract. The tissue closure device 402 of this embodiment may include a body portion 404 and first and second end portions 406, 408 having some or all of the features of the embodiments described in relation to FIGS. 4A-4E. In some embodiments the tissue closure device includes third and fourth end portions 416, 418 disposed at an end of the body opposite that of the first and second end portions 406, 408. The third and fourth end portions 416, 418 may be folded backward so that they lie approximately parallel to an outer surface of the body portion 404. The first and second end portions 406, 408 may folded forward so that they lie approximately parallel to an outer surface of the body portion 404. Thus arranged, distal portions 416B, 418B of the third and fourth end portions 416, 418 may point in a direction opposite to that of the distal portions 406B, 408B of the first and second end portions 406, 408.

The tissue closure device 402 can be positioned so the distal portions 406B, 408B are positioned adjacent a distal end 412A of the lumen 412. The tissue closure device 402 may also be generally oriented along the longitudinal axis X-X of the lumen 412 to enable the user to direct the tissue closure device 402 toward the target tissue site. To engage the tissue closure device 402 with a target tissue portion 414, the distal end 415A of the needle 415 may be moved into engagement with the target tissue portion 414 to pierce target tissue portion. A pusher (not shown) disposed within the needle 415 may be used to move the tissue closure device 402 in the direction of arrow “A” out of the lumen 412 so that the distal portions 406B, 408B of the first and second end portions 406, 408 pass through the target tissue portion. As the distal portions 406B, 408B of the first and second end portions 406, 408 pass through the target tissue portion the bias in the first and second end portions 406, 408 causes the distal portions 406B, 408B to rotate (or otherwise move) away from their respective proximal portions 406A, 408A to engage a distal side 414C of the target tissue portion 414. When the tissue closure device 402 is completely expelled from the lumen 412, the third and fourth end portions may rotate (or otherwise move) in an opposite direction to that of the first and second end portions 406, 408 to engage a proximal side 414D of the target tissue portion 414, thus coupling the tissue closure device 402 to the target tissue portion.

Although the illustrated embodiment shows the tissue closure device 402 deployed with the use of a needle 415, it will be appreciated that an endoscope could also be used. Deployment of the tissue closure device 402, either via needle or endoscope, can be achieved using of an appropriate pusher (not shown) disposed behind the tissue closure device 402 within the lumen 412. The user can apply an appropriate force to the pusher to move the proximal portions 406A, 408A of the first and second end portions 406, 408 out of the distal end 412A of the lumen 412 to allow the distal portions 406B, 408B to engage the first and second target tissue portions 414A, 414B. The endoscope may then be removed, leaving the tissue closure device 402 coupled to the target tissue portion 414.

Although the tissue closure device 402 has been described as having first and second end portions 406, 408, it will be appreciated that fewer or greater numbers of end portions can be used to couple the body portion 404 to the target tissue.

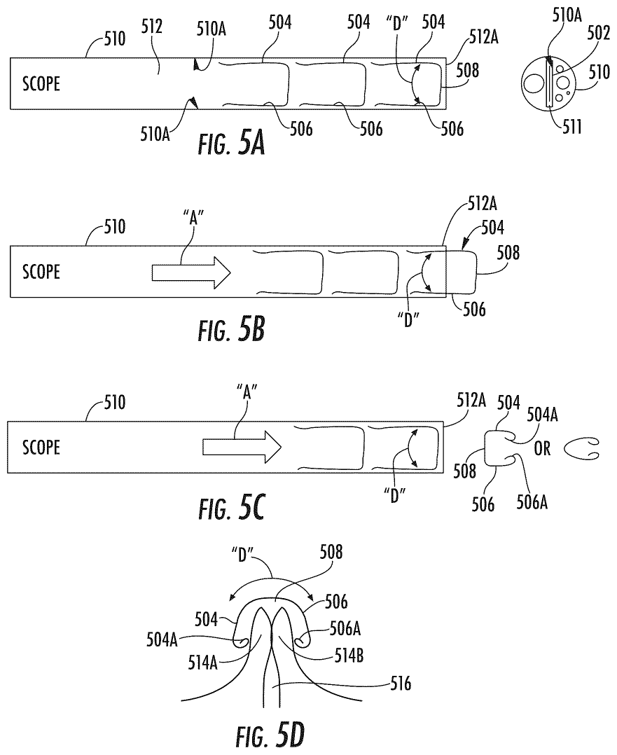

An embodiment of a tissue closure device 502 is illustrated in FIGS. 5A-5D. The tissue closure device 502 includes first and second end portions 504, 506 and a body portion 508 that connects the first end portion to the second end portion. In some nonlimiting example embodiments the first and second end portions 504, 506 and the body portion 508 comprise a single element made from a super-elastic material such as Nitinol. In other embodiments the first and second end portions 504, 506 can be super-elastic elements, while the body portion 508 can be made from a different material coupled to the first and second end portions.

As mentioned, the first and second end portions 504, 506 may be comprised of a resilient material such that the first and second end portions function as spring elements. In some embodiments the first and second end portions 504, 506 are be biased toward each other when the tissue closure device 502 is in a deployed configuration. Thus, when the first end portion 504 is engaged with a first target tissue portion 514A and the second end portion 506 is engaged with a second target tissue portion 514B, the spring bias in the first and second end portions urges the first and second target tissue portions together to close an opening 516 in the tissue.

The first and second end portions 504, 506 may each have a tissue engaging end 504A, 506A for engaging and retaining the target tissue portions 514A, 514B to the tissue closure device 502. The illustrated embodiment shows tissue engaging ends 504A, 506A having a curved or curled configuration. The tissue engaging ends 504A, 506A could, alternatively, include barbs, hooks, or the like.

The tissue closure device 502 may be delivered to the target tissue site in a first configuration, which is shown in FIGS. 5A-5C. In this configuration, the first and second end portions 504, 506 have been rotated (or otherwise moved) away from each other and disposed within the lumen 512 of the endoscope 510 such that the first and second end portions 504, 506 are oriented away from a distal end 512A of the lumen 512 and the body portion 508 is oriented toward the distal end of the lumen. In this configuration, due to the spring bias in the tissue closure device 502 the first and second end portions 504, 506 are biased away from each other and into engagement with an inner surface 510A of the endoscope 510. In some embodiments the tissue closure device 502 can be positioned within a slot 511 in the endoscope 510, which maintains the tissue closure device 502 in the illustrated first configuration prior to deployment.

The tissue closure device 502 may be deployed from the distal end 512A of the lumen 512 of the endoscope 510 to enable the first and second end portions 504, 506 to engage the first and second target tissue portions 514A, 514B. Deployment can be achieved using an appropriate pusher (not shown) positioned within the lumen 512, behind the tissue closure device 502 (or series of tissue closure devices). The user can apply an appropriate force to the pusher to move the tissue closure device 502 in the direction of arrow “A” out of the distal end 512A of the lumen 512, thereby releasing the first and second end portions 504, 506 such that they rotate (or otherwise move) in the direction of arrow “D” to assume a second configuration, shown in FIG. 5D. As can be seen, in the second configuration the tissue engaging ends 504A, 506A of the first and second end portions 504, 506 engage the first and second target tissue portions 514A, 514B and press the tissue portions together. Although the tissue closure device 502 is illustrated as being generally U-shaped, it will be appreciation that the tissue closure device may be provided in a variety of different shapes, including V-shaped.

In some embodiments the tissue engaging ends 504A, 506A of the first and second end portions 504, 506 may engage the first and second target tissue portions 514A, 514B, draw them together and hold them in position to close the opening 516. In other embodiments a separate gripping device (not shown) may be used to draw the first and second target tissue portions 514A, 514B together so that the tissue closure device 502, or series of tissue closure devices, can be employed to clamp the first and second target tissue portions 514A, 514B together.

As shown in FIGS. 5A-5C, a plurality of tissue closure devices 502 may be used to close an opening 216 in the tissue. Where a plurality of tissue closure devices 502 are employed, such tissue closure devices may be pre-loaded within the lumen 512 in the endoscope 510 and sequentially deployed using a pusher disposed behind the pre-loaded tissue closure devices. Alternatively, individual tissue closure devices 502 may be loaded into the lumen 512 and deployed individually.

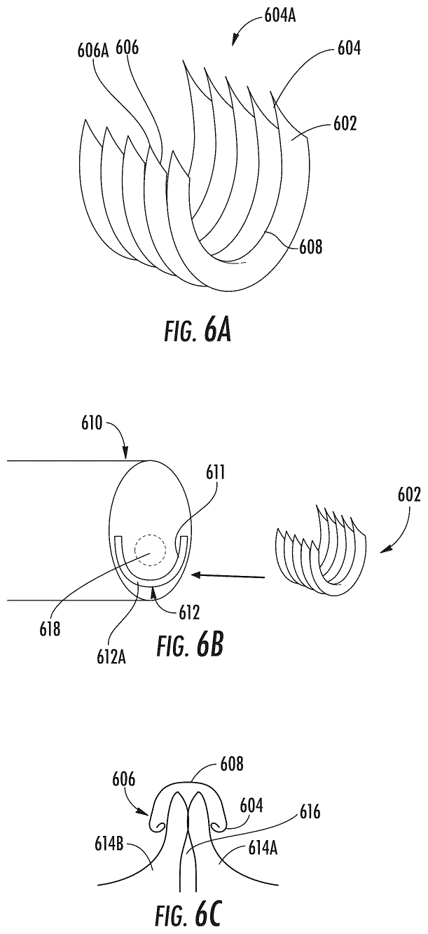

An embodiment of a tissue closure device 602 is illustrated in FIGS. 6A-6C. FIG. 6A shows a plurality of tissue closure device 602 assembled together as a “clip” of tissue closure devices, which can be loaded as a single unit into a lumen 612 of an endoscope 610.

The tissue closure device 602 of this embodiment can include first and second end portions 604, 606 and a body portion 608 that connects the first end portion to the second end portion. In some nonlimiting example embodiments the first and second end portions 604, 606 and the body portion 608 comprise super-elastic elements such as Nitinol, although other materials can be used such as appropriate metals and/or polymers.

As mentioned, the first and second end portions 604, 606 may be comprised of a resilient material such that the first and second end portions can function as spring elements. As such, when the first end portion 604 of the tissue closure device 602 is deployed from the lumen 612 and engaged with a first target tissue portion 614A and the second end portion 606 is similarly deployed and engaged with a second target tissue portion 614B, the first and second end portions urge the first and second target tissue portions together to close an opening 616 in the tissue.

The first and second end portions 604, 606 may each have a tissue engaging end 604A, 606A for retaining the first and second target tissue portions 614A, 614B to the tissue closure device 602. The illustrated embodiment shows tissue engaging ends 604A, 606A having sharpened tips for piercing tissue, though it will be appreciated that the tissue engaging ends may have other shapes such as hooks, barbs, and the like, or atraumatic, blunt, or rounded tips.

The tissue closure device 602 may be delivered to the target tissue site in a first, expanded, configuration. In this configuration, the first and second end portions 604, 606 have been rotated (or otherwise moved) away from each other and disposed within a lumen 612 of the endoscope 610 which holds them in the first configuration. In the illustrated embodiment the lumen 612 is an arc-shaped slot that has an arc radius that is larger than the unconstrained radius of the tissue closure device 602. The lumen 612 may retain the closure device 602, which may extend through a flexible tube of the endoscope to the proximal end, although it is understood that the distal end of the endoscope 610 may include a compartment or recess for receiving closure device 602. As mentioned, in this configuration, due to the spring bias in the tissue closure device 602 the first and second end portions 604, 606 are biased inward, toward each other, and into engagement with an inner surface 611 of the endoscope 610.

The tissue closure device 602 may be deployed from the distal end 612A of the lumen 612 of the endoscope 610 to enable the first and second end portions 604, 606 to engage the first and second target tissue portions 614A, 614B. Deployment can be achieved using an appropriate pusher (not shown) positioned within the lumen 612, behind the tissue closure device 602 (or clip of tissue closure devices, as shown). The user can apply an appropriate force to the pusher to move the tissue closure device 602 out of the distal end 612A of the lumen 612, thereby releasing the first and second end portions 604, 606 such that they move toward each other. As can be seen, in the second configuration the tissue engaging ends 604A, 606A of the first and second end portions 604, 606 engage the first and second target tissue portions 614A, 614B and press the tissue portions together.

In some embodiments the tissue engaging ends 604A, 606A of the first and second end portions 604, 606 may engage the first and second target tissue portions 614A, 614B, to both draw them together and hold them in position to close the opening 616. In other embodiments a separate gripping device (not shown) may be used to draw the first and second target tissue portions 614A, 614B together so that the tissue closure device 602 can be employed to hold the tissue portions 614A, 614B together.

In some embodiments, the endoscope 618 may include a working channel 618 that can be used to apply suction, deploy a tissue grasping tool, and the like.

In some embodiments the tissue closure device 602 can be configured so that one of the first and second end portions 604, 606 is positioned closer to the distal end 612A of the lumen 612 compared to the other of the first and second end portions. Such an arrangement can permit one of the first and second end portions 604, 606 to deploy before the other as the tissue closure device 602 is being deployed from the lumen 612. This, in turn, can allow the user to engage one of the first and second target tissue portions 614A, 614B with one of the tissue engaging ends 604A, 606A ensure the correct area has been acquired or engaged. The user can then apply force to the endoscope 610 to pull the captured tissue to close the opening 616 between the first and second target tissue portions 614A, 614B before deploying the other one of the first and second end portions 604, 606 to clamp the tissue portions together.

As shown, a plurality of closure devices 602 may be provided in a clip or cartridge (FIG. 6A). The plurality of tissue closure devices 602 may be used to close openings 616 of various sizes. Where a plurality of tissue closure devices 602 are employed, such tissue closure devices may be pre-loaded within the lumen 612 in the endoscope 610 and sequentially deployed using a pusher disposed behind the pre-loaded tissue closure devices. Alternatively, individual tissue closure devices 602 may be loaded into the lumen 612 and deployed individually.

For embodiments in which the plurality of closure devices 602 are provided in a clip or cartridge, a spacer may be provided between adjacent clips. The spacer could allow full clip deployment without exposing (and potentially partially deploying) the subsequent clip. The spacer could be made from any biocompatible material including biodegradables. The spacer(s) could be removed after the closure is complete or could be allowed to pass naturally through the gastro-intestinal tract.

Devices according to the embodiments described, and in accordance with other embodiments of the present disclosure, alone or in a system or kit or as part of a method or procedure, including with other accessories, may be used in cavities, lumens, tracts, vessels, and organs of the body, etc.

Variations, modifications, and other implementations of the present disclosure in addition to the various embodiments described herein will occur to those of ordinary skill in the art. Accordingly, the present disclosure is to be defined not by the preceding illustrative description but instead by the following claims.

What is claimed is:

1. A tissue closure system for sealing an opening between a first tissue portion and a second tissue portion, said tissue closure system comprising:
a delivery device comprising a tubular element with a lumen therethrough; and
a tissue closure device positioned within said delivery device lumen, said tissue closure device configured to transition between a first configuration when positioned within said delivery device lumen and a second configuration when positioned outside said delivery device lumen, said tissue closure device comprising:
a body portion having a first end and a second end;
a first end portion having a first end coupled to said first end of said body portion, and a free end; and
a second end portion having a first end coupled to said second end of said body portion, and a free end;
wherein:
said first end portion transitions to an unconstrained configuration when moved out of said delivery device lumen to engage the first tissue portion with said free end thereof adjacent a central portion of said body portion and;
said second end portion transitions to an unconstrained configuration when moved out of said delivery device lumen to engage the second tissue portion with said free end thereof adjacent a central portion of said body portion; and
said first end portion and said second end portion, in respective unconstrained configurations, are biased toward each other to draw together the first tissue portion engaged by said first end portion and the second tissue portion engaged by said second end portion to seal the opening in the tissue.

2. The tissue closure system of claim 1, wherein said first end portion and said second end portion are formed from a shape memory material to form spring biased elements when in respective unconstrained configurations biasing the first tissue portion and the second tissue portion together to seal the opening in the tissue.

3. The tissue closure system of claim 2, wherein said body portion is substantially straight when moved out of said delivery device and extended through the first tissue portion and the second tissue portion.

4. The tissue closure system of claim 3, wherein said first end portion and said second end portion in respective unconstrained configurations each form a wave or loop shape each providing a spring bias to urge the first tissue portion and the second tissue portion together to seal the opening in the tissue.

5. The tissue closure system of claim 1, wherein said free end of said first end portion is shaped to penetrate tissue as it is moved out of the delivery device lumen.

6. The tissue closure system of claim 5, further comprising a pusher within said delivery device lumen movable to push said tissue closure device out of said delivery device lumen to pass said free end of said first end portion through the first tissue portion and the second tissue portion.

7. The tissue closure system of claim 1, wherein said body portion comprises a suture formed separately from and connected to said first end portion and said second end portion.

8. The tissue closure system of claim 7, wherein said first end portion and said second end portion each comprise an elastic tubular element.

9. The tissue closure system of claim 1, wherein said delivery device has a tip configured to penetrate through the first tissue portion and the second tissue portion.

10. The tissue closure system of claim 1, wherein more than one tissue closure device is preloaded within said delivery device.

11. A method of closing an opening between a first tissue portion and a second tissue portion, said method comprising:
delivering a tissue closure device to the first tissue portion in a constrained configuration within a delivery device lumen, the tissue closure device having a first end portion with a first end and a free end, a second end portion with a first end and a free end, and a body portion with a first end coupled to the first end of the first end portion and a second end coupled to the first end of the second end portion;
penetrating the first tissue portion and the second tissue portion to extend the tissue closure device therethrough;
extending the first end portion through the penetrated first tissue portion and the penetrated second tissue portion; and
releasing the first end portion to transition from the constrained configuration to an unconstrained configuration engaging the first tissue portion with the free end thereof extended toward a central portion of the body portion, and releasing the second end portion to transition from the constrained configuration to an unconstrained configuration engaging the second tissue portion with the free end extended toward a central portion of the body portion, thereby urging the first tissue portion and the second tissue portion together to seal the opening.

12. The method of claim 11, wherein a tissue-penetrating tip is formed on the free end of the first end portion, said method further comprising penetrating the first tissue portion and the second tissue portion with the tissue-penetrating tip formed on the free end of the first end portion.

13. The method of claim 11, further comprising penetrating the first tissue portion and the second tissue portion with a tissue-penetrating tip formed on a sheath in which the tissue closure device is housed.

14. The method of claim 11, wherein releasing the first end portion further comprises allowing the first end portion to transition to the unconstrained configuration with the free end thereof adjacent the central portion of the body portion.

15. The method of claim 14, wherein releasing the second end portion further comprises allowing the second end portion to transition to the unconstrained configuration with the free end thereof adjacent the central portion of the body portion, so that the first tissue portion and the second tissue portion are urged together by the first end portion and the second end portion, respectively, to seal the opening.

16. The method of claim 11, further comprising delivering a plurality of tissue closure device sequentially.

17. A tissue closure device for sealing an opening between a first tissue portion and a second tissue portion, said tissue closure device comprising:
a body portion having a first end and a second end;
a first end portion having a first end coupled to said first end of said body portion, and a free end; and
a second end portion having a first end coupled to said second end of the body portion, and a free end;
wherein:
said first end portion is configured to transition from a constrained configuration to an unconstrained configuration engaging the first tissue portion;
said second end portion is configured to transition from a constrained configuration to an unconstrained configuration engaging the second tissue portion; and
in the respective unconstrained configurations of said first end portion and said second end portion, said free end of said first end portion and said free end of said second end portion are disposed adjacent to a central portion of said body portion to urge together the first tissue portion and the second tissue portion positioned therebetween to seal the opening.

18. The tissue closure device of claim 17, wherein said first end portion and said second end portion are substantially elongated when in the unconstrained configuration.

19. The tissue closure device of claim 17, wherein said body portion is substantially straight when said tissue closure device is in the constrained configuration and when said tissue closure device is in the unconstrained configuration.

20. The tissue closure device of claim 17, wherein said free end of said first end portion is shaped to penetrate tissue.
